(19) United States
(12) Patent Application Publication
(10) Pub. No.: US 2025/0367357 A1
(43) Pub. Date: Dec. 4, 2025

(54) RESIN, ADSORPTION MATERIAL, AND METHOD OF LOWERING LDL-C CONCENTRATION IN BLOOD

(71) Applicant: INDUSTRIAL TECHNOLOGY RESEARCH INSTITUTE, Hsinchu (TW)

(72) Inventors: Ping-Yen CHEN, Hsinchu City (TW); Min-Fei TSAI, Taoyuan City (TW); Li-Han CHUNG, Miaoli City (TW); Yen-Cheng LI, Hsinchu City (TW)

(73) Assignee: INDUSTRIAL TECHNOLOGY RESEARCH INSTITUTE, Hsinchu (TW)

(21) Appl. No.: 18/951,022

(22) Filed: Nov. 18, 2024

(30) Foreign Application Priority Data
May 30, 2024 (TW) 113120100

(51) Int. Cl. A61M 1/36; C08G 75/23

(52) U.S. Cl. CPC A61M 1/3679; C08G 75/23; A61M 2202/08

(57) ABSTRACT

A resin is formed through a ring-opening reaction of dextran sulfate sodium salt and a modified polyether sulfone. The modified polyether sulfone is formed by a reaction of polyether sulfone and an epoxy compound containing a carbon-carbon double bond. The resin may serve as an adsorption material, which can be a porous film or a porous hollow fiber. The adsorption material can be in contact with blood to lower LDL-C concentration in the blood.

### CROSS REFERENCE TO RELATED APPLICATIONS

The present application is based on, and claims priority from, Taiwan Application Serial Number 113120100, filed on May 30, 2024, the disclosure of which is hereby incorporated by reference herein in its entirety.

### TECHNICAL FIELD

The technical field relates to a method of lowering low-density lipoprotein cholesterol (LDL-C) concentration in blood, and in particular it relates to an adsorption material for the method.

### BACKGROUND

High cholesterol in the blood is a major risk to health, potentially inducing atherosclerotic diseases in humans. Common atherosclerotic diseases include coronary heart disease (e.g., myocardial infarction, angina pectoris, and sudden death), cerebral infarction, and peripheral vascular thromboembolic disease. The latest international medical treatment recommendations require positive control of cholesterol in high-risk patients. In particular, high-risk patients with coronary artery disease, acute myocardial infarction, ischemic stroke, diabetes, and chronic kidney disease should pay special attention to low-density lipoprotein cholesterol (LDL-C) of cholesterol.

Clinically, lipid-lowering drugs (e.g., statins) are mainly used to control cholesterol concentration now. In addition to using drugs to control cholesterol concentration, blood purification treatment such as LDL-C lowering technology (LDL-C apheresis) is used. The blood is drawn out of the body and passes through an adsorption column made of special material that can adsorb LDL-C to quickly and effectively lower the LDL-C concentration in the blood. In the past, LDL-C lowering technology (e.g., LDL-C apheresis) was mainly used to treat patients with family hypercholesterolemia, whose LDL-C concentration of blood is 300 mg/dL to 600 mg/dL or even higher and cannot be controlled by lipid-lowering drugs. The lipid-lowering drugs may have side effects such as soaring liver index, dizziness, vomiting, and the like to some patients with high cholesterol, such that LDL-C lowering technology can be used instead to purify the blood.

Accordingly, a novel adsorption material is called-for to purify the blood with an overly high concentration of LDL-C.

### SUMMARY

One embodiment of the disclosure provides a resin formed through a ring-opening reaction of dextran sulfate sodium salt and a modified polyether sulfone. The modified polyether sulfone is formed by a reaction of polyether sulfone and an epoxy compound containing a carbon-carbon double bond.

In some embodiments, the polyether sulfone and the epoxy compound containing a carbon-carbon double bond have a weight ratio of 100:20 to 100:30.

In some embodiments, the modified polyether sulfone and the dextran sulfate sodium salt have a weight ratio of 100:1 to 100:1500.

In some embodiments, a repeating unit of the polyether sulfone is

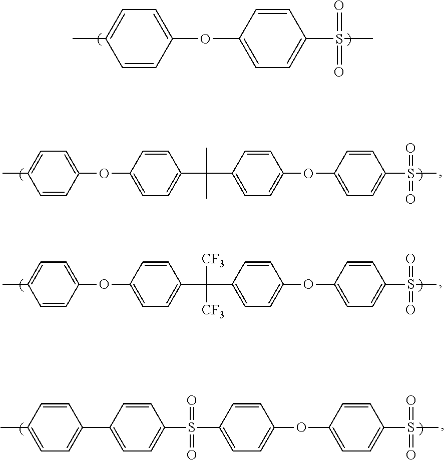

or a combination thereof.

In some embodiments, the polyether sulfone has a weight average molecular weight of 55,000 g/mol to 75,000 g/mol.

In some embodiments, the dextran sulfate sodium salt has a weight average molecular weight of 5000 g/mol to 10000 g/mol.

In some embodiments, the epoxy compound containing a carbon-carbon double bond is

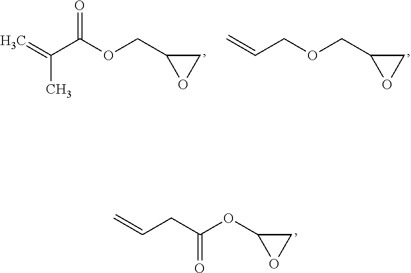

or a combination thereof.

One embodiment of the disclosure provides an adsorption material, including the described resin, wherein the adsorption material is in the form of a porous film or a porous hollow fiber.

In some embodiments, the adsorption material has a porosity of 60% to 90%.

In some embodiments, the porous film has a thickness of 20 micrometers to 60 micrometers.

In some embodiments, the porous hollow fiber has an inner diameter of 300 micrometers to 600 micrometers, and the porous hollow fiber has a thickness of 80 micrometers to 150 micrometers.

One embodiment of the disclosure provides a method of lowering low-density lipoprotein cholesterol concentration in the blood, including bringing the blood into contact with the aforementioned adsorption material to lower the low-density lipoprotein cholesterol concentration in the blood.

A detailed description is given in the following embodiments.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

One embodiment of the disclosure provides a resin formed through a ring-opening reaction of dextran sulfate sodium salt and a modified polyether sulfone. The modified polyether sulfone is formed by a reaction of polyether sulfone and an epoxy compound containing a carbon-carbon double bond, e.g., by the reaction under UV irradiation. For example, the polyether sulfone and the epoxy compound containing a carbon-carbon double bond are dissolved in a solvent, and the solution was irradiated by UV, such that the polyether sulfone and the epoxy compound containing a carbon-carbon double bond react to form the modified polyether sulfone. According to the spectral identification (e.g., NMR, IR, PGC-MS, or the like) of the modified polyether sulfone, the spectra show that epoxy groups exist but without carbon-carbon double bonds. It means that the epoxy compound containing a carbon-carbon double bond and the polyether sulfone react to form a chemical bond under the UV irradiation, i.e. forming the modified polyether sulfone rather than a mixture of the polyether sulfone and the epoxy compound containing a carbon-carbon double bond. In some embodiments, the polyether sulfone and the epoxy compound containing a carbon-carbon double bond respectively form radicals in the reaction, and the radicals form the chemical bonding to generate the modified polyether sulfone.

In some embodiments, the solvent for dissolving the polyether sulfone and the epoxy compound containing a carbon-carbon double bond can be N-methylpyrrolidone, dimethylformamide, dimethylacetamide, dimethyl sulfoxide, or a combination thereof.

In some embodiments, a repeating unit of the polyether sulfone is

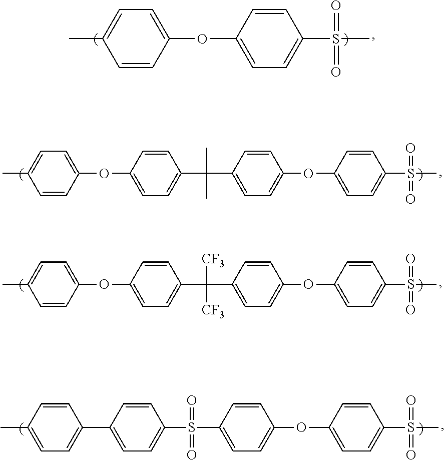

or a combination thereof. In some embodiments, the polyether sulfone has a weight average molecular weight of 55,000 g/mol to 75,000 g/mol. If the weight average molecular weight of the polyether sulfone is too low, the resin cannot be processed to form a porous film. If the weight average molecular weight of the polyether sulfone is too high, the polyether sulfone cannot be efficiently modified.

In some embodiments, the epoxy compound containing a carbon-carbon double bond is

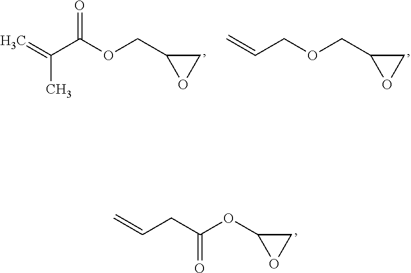

or a combination thereof.

In some embodiments, the polyether sulfone and the epoxy compound containing a carbon-carbon double bond have a weight ratio of 100:20 to 100:30. If the amount of the epoxy compound containing a carbon-carbon double bond is too low, the modification ratio of the polyether sulfone will be too low, and the modified polyether sulfone and the original polyether sulfone will be free of difference as identified by spectra. If the amount of the epoxy compound containing a carbon-carbon double bond is too high, it may generate a gel of the epoxy compound containing a carbon-carbon double bond. Note that the amount of the epoxy compound containing a carbon-carbon double bond used in the reaction is possibly excessive, and the unreacted epoxy compound containing a carbon-carbon double bond will be washed away after the reaction. In other words, the aforementioned ratio is not the weight ratio of reactants; it is the weight ratio of the polyether sulfone part (for the structure in the modified polyether sulfone corresponding to the polyether sulfone) and the epoxy compound containing a carbon-carbon double bond part (for the structure in the modified polyether sulfone corresponding to the epoxy compound containing a carbon-carbon double bond) in the product (the modified polyether sulfone).

In some embodiments, the modified polyether sulfone is processed and forms a porous film or a porous hollow fiber, which is then dipped into an alkaline aqueous solution of dextran sulfate sodium salt. As such, the dextran sulfate sodium salt and the epoxy group of the modified polyether sulfone will react to perform a ring-opening reaction, thereby forming a resin (i.e., the dextran sulfate sodium salt is grafted to the modified polyether sulfone). As such, an adsorption material of the resin is obtained, and the adsorption material is in the form of the porous film or the porous hollow fiber. Note that in the disclosure, the porous film or the porous hollow fiber of the modified polyether sulfone is prepared first, and the dextran sulfate sodium salt is then grafted onto the surface of the porous film or the porous hollow fiber. The disclosure does not graft the dextran sulfate sodium salt to the modified polyether sulfone (to form the resin), and then process the material to form a porous film or a porous hollow fiber. As a result, it can be ensured that the dextran sulfate sodium salt is grafted on the surface of the porous film or the porous hollow fiber. The dextran sulfate sodium salt will be exposed on the surface but not wrapped in the material of the porous film or the porous hollow fiber.

In some embodiments, the modified polyether sulfone and the dextran sulfate sodium salt have a weight ratio of 100:1 to 100:1500. If the amount of the dextran sulfate sodium salt is too low, the effect of adsorbing LDL-C will be poor (e.g., LDL-C lowering ratio<3%). If the amount of the dextran sulfate sodium salt is too high, the raw material of the dextran sulfate sodium salt will be wasted, and the excessive raw material of the dextran sulfate sodium salt will increase the production cost. Note that the amount of the dextran sulfate sodium salt used in the reaction is possibly excessive, and the unreacted dextran sulfate sodium salt will be washed away after the reaction. In other words, the aforementioned ratio is not the weight ratio of reactants; it is the weight ratio of the modified polyether sulfone part (for the structure in the resin corresponding to the modified polyether sulfone) and the dextran sulfate sodium salt part (for the structure in the resin corresponding to the dextran sulfate sodium salt) in the product (the resin).

In some embodiments, the dextran sulfate sodium salt has a weight average molecular weight of 5000 g/mol to 10000 g/mol. If the weight average molecular weight of the dextran sulfate sodium salt is too low, the charge adsorption ability of the molecular chain of the dextran sulfate sodium salt will be lowered, and the effect of adsorbing LDL-C will be poor (e.g., LDL-C lowering ratio<3%). If the weight average molecular weight of the dextran sulfate sodium salt is too high, the charge adsorption ability of the molecular chain of the dextran sulfate sodium salt will be lowered or the ring-opening and grafting will be affected by steric hindrance of the molecular chain of the dextran sulfate sodium salt, and the effect of adsorbing LDL-C will be poor (e.g., LDL-C lowering ratio<3%).

One embodiment of the disclosure provides an adsorption material including the described resin. The adsorption material is in the form of a porous film or a porous hollow fiber. In some embodiments, the adsorption material has a porosity of 60% to 90%. If the porosity of the adsorption material is too low, the surface area of the hollow fiber or the film will be too low, thereby lowering the adsorption effect due to insufficient contact area. If the porosity of the adsorption material is too high, the mechanical and physical properties of the hollow fiber or the film will be poor, resulting in difficulty in maintaining its dimension and appearance and prone to broken foams. In some embodiments, the porous film has a thickness of 20 micrometers to 60 micrometers. If the porous film is too thin, the mechanical and physical properties of the porous film will be poor, resulting in difficulty in maintaining its dimension and appearance and prone to broken foams. If the porous film is too thick, its efficiency of filtration or penetration will be poor. In some embodiments, the porous hollow fiber has an inner diameter of 300 micrometers to 600 micrometers, and the porous hollow fiber has a thickness of 80 micrometers to 150 micrometers. If the inner diameter of the porous hollow fiber is too small, the adsorption material may result in the rupture of blood cells and hemolysis during the LDL-C concentration lowering therapy (e.g., LDL-C apheresis) in the blood. If the inner diameter of the porous hollow fiber is too large, the bundle will be easily deformed, and the effective contact surface of the cross-sectional area of the bundle will be decreased. If the thickness of the porous hollow fiber is too small, the mechanical and physical properties of the porous hollow fiber will be poor, resulting in difficulty in maintaining its dimension and appearance and prone to broken foams. If the thickness of the porous hollow fiber is too large, the porosity of the porous structure of the fiber will be decreased to lower the penetration effect.

One embodiment of the disclosure provides a method of lowering low-density lipoprotein cholesterol concentration in the blood, including bringing the blood into contact with the aforementioned adsorption material to lower low-density lipoprotein cholesterol concentration in the blood.

Accordingly, the disclosure provides the adsorption material and the resin for the adsorption material, which may efficiently lower LDL-C concentration in the blood. In addition, the method of lowering the LDL-C concentration in the blood can be combined with other conventional hemodialysis (e.g. kidney dialysis) to further save treatment time of the patients.

Below, exemplary embodiments will be described in detail so as to be easily realized by a person having ordinary knowledge in the art. The inventive concept may be embodied in various forms without being limited to the exemplary embodiments set forth herein. Descriptions of well-known parts are omitted for clarity.

### EXAMPLES

### Example 1 (Modified Polyether Sulfone)

10 g of polyether sulfone (PES, Ultrason® E6020P commercially available from BASF) and 20 g of glycidyl methacrylate (GMA) were added into 100 mL of N-methyl pyrrolidone (NMP) and stirred to be evenly dissolved. The solution was continuously bubbled with nitrogen for 1 hour to remove oxygen in the solution. The solution was then irradiated by UV radiation (UVC, 254 nm and 125 mW/cm²; UVA 365 nm and 150 mW/cm²) for 2 minutes, and then bubbled with nitrogen again at room temperature to stir and react for 5.5 hours, such that PES and GMA reacted to form modified PES. The solution was poured into water to precipitate a white solid. The white solid was washed by ethanol to remove the residual solvent and the unreacted GMA monomer. The washed solid was dried at 80° C. to obtain the modified PES. According to the calculation result from the NMR spectrum (Brucker 400 MHz, DMSO-d₆) of the modified PES, the repeating units of PES and GMA had a molar ratio of 75:25, i.e. PES and GMA had a weight ratio of 100:20.4.

### Example 2 (Porous Film of the Modified PES)

16 parts by weight of the modified PES in Example 1, 1.6 parts by weight of the polyvinylpyrrolidone (PVP40k, commercially available from SIGMA) serving as porogen, 43 parts by weight of NMP, and 43 parts by weight of polyethylene glycol (PEG600, commercially available from SIGMA) were mixed to form a polymer mixture solution. The polymer mixture solution was uniformly coated on a glass plate by a metal blade to form a coating layer on the glass plate, and the blade gap was 100 micrometers. The glass plate and the coating layer were then dipped into a co-solvent of NMP/water (w/w=70/30) at 25° C. for 1 minute, and then dipped into water at 25° C. to remove PEG, PVP, and NMP in the coating layer, thereby forming a film with multiple micropores. The film had a thickness of 60 micrometers.

### Example 3 (Porous Hollow Fiber of the Modified PES)

16 parts by weight of the modified PES in Example 1, 1.6 parts by weight of PVP40k serving as porogen, 43 parts by weight of NMP, and 43 parts by weight of PEG600 were mixed to form a polymer mixture solution. The polymer mixture solution was stood in a spinning feed tank for at least 18 hours to remove bubbles in the solution. The temperatures of the spinning feed tank and a water bath tank were maintained at 25° C. by a thermostat. The feeding pressure was 2.5 atm, the core liquid was NMP/water (w/w=3/1), the surrounding temperature of the spinneret was 25° C., and the humidity was 90% RH. The obtained porous hollow fiber had an inner diameter of about 450 micrometers and a thickness of about 100 micrometers.

### Comparative Example 1 (Hollow Fiber of PES)

16 parts by weight of PES, 1.6 parts by weight of PVP40k serving as porogen, 43 parts by weight of NMP, and 43 parts by weight of PEG600 were mixed to form a polymer mixture solution. The polymer mixture solution was stood in a spinning feed tank for at least 18 hours to remove bubbles in the solution. The temperatures of the spinning feed tank and a water bath tank were maintained at 25° C. by a thermostat. The feeding pressure was 2.5 atm, the core liquid was NMP/water (w/w=3/1), the surrounding temperature of the spinneret was 25° C., and the humidity was 90% RH. The obtained porous hollow fiber had an inner diameter of about 450 micrometers and a thickness of about 100 micrometers.

### Comparative Example 2 (Hollow Fiber of PES and DS Mixture)

16 parts by weight of PES, 0.16 parts by weight of dextran sulfate sodium salt (DS, MW=8000 g/mol, commercially available from SIGMA), 1.6 parts by weight of PVP40k serving as porogen, 43 parts by weight of NMP, and 43 parts by weight of PEG600 were mixed to form a polymer mixture solution. The polymer mixture solution was stood in a spinning feed tank for at least 18 hours to remove bubbles in the solution. The temperatures of the spinning feed tank and a water bath tank were maintained at 25° C. by a thermostat. The feeding pressure was 2.5 atm, the core liquid was NMP/water (w/w=3/1), the surrounding temperature of the spinneret was 25° C., and the humidity was 90% RH. The obtained porous hollow fiber had an inner diameter of about 450 micrometers and a thickness of about 100 micrometers.

### Example 4 (Film of Resin Formed by Reacting DS and the Modified PES)

The porous film of the modified PES in Example 2 was dipped into 100 mL of an aqueous solution of sodium hydroxide (0.1 M) and DS (5%, MW=8000 g/mol, commercially available from SIGMA). The aqueous solution was then heated to 60° C. and stirred for 24 hours, such that epoxy groups of the modified PES and DS reacted to perform a ring-opening reaction to form a resin. The film of the resin was then washed by de-ionized water to neutralize the film. Subsequently, the film of the resin was dipped into deionized water to store the film. As known from titration method, the modified PES and DS in the resin have a weight ratio of 100:1.6. The film had a porosity of 85%, which was determined by calculating XCT (X-ray Computed Tomography) images and SEM cross-sectional images. XCT combines computing technology and X-ray radiology to analyze the porosity of the porous material.

### Example 5 (Film of Resin Formed by Reacting DS and the Modified PES)

The porous film of the modified PES in Example 2 was dipped into 100 mL of an aqueous solution of sodium hydroxide (0.1 M) and DS (15%, MW=8000 g/mol, commercially available from SIGMA). The aqueous solution was then heated to 60° C. and stirred for 24 hours, such that epoxy groups of the modified PES and DS reacted to perform a ring-opening reaction to form a resin. The film of the resin was then washed by de-ionized water to neutralize the film. Subsequently, the film of the resin was dipped into deionized water to store the film. As known from titration method, the modified PES and DS in the resin have a weight ratio of 100:3.2. The film had a porosity of 85%, which was determined by calculating XCT images and SEM cross-sectional images.

### Example 6 (Lowering LDL-C Concentration in Serum by the Film of the Resin)

The film of the resin in Example 4 (having an area of about 16 cm²) was cut to little pieces (having an area of about 5 mm*5 mm). The little pieces were then put into 6 mL of porcine serum (commercially available from Gibco™), and the mixture was placed on a shaking platform for 4 hours to adsorb cholesterol from the porcine serum. The upper clear liquid was then taken to analyze the composition of the treated porcine serum (Measured by Direct Homogeneous Surfactant (ADVIA 1800, ADVIA Chemistry XPT, Dimension EXL)), as tabulated in Table 1. The above test was repeated, and the difference was the film being changed to the film of the resin in Example 5.

TABLE 1

| | | High-density | Low-density | |
|---|---|---|---|---|
| | Total | lipoprotein | lipoprotein | |
| | cholesterol | cholesterol | cholesterol | Triglyceride |
| | (TCO, mg/dL) | (HDL, mg/dL) | (LDL-C, mg/dL) | (TG, mg/dL) |
| Non-treated | 103 | 26 | 51 | 59 |
| porcine serum | | | | |
| Porcine serum after | 86 | 22 | 41 | 35 |
| being treated by the | | | | |
| film of the resin | | | | |
| in Example 4 | | | | |
| Porcine serum after | 85 | 23 | 40 | 33 |
| being treated by the | | | | |
| film of the resin | | | | |
| in Example 5 | | | | |

As shown in Table 1, the films of the resin in Example 4 or 5 could efficiently lower LDL-C concentration in the porcine serum.

### Example 7 (Hollow Fiber of Resin Formed by Reacting DS and the Modified PES)

The hollow fiber of the modified PES in Example 3 was dipped into 150 mL of an aqueous solution of sodium hydroxide (0.1 M) and DS (15%, MW=8000 g/mol, commercially available from SIGMA). The aqueous solution was then heated to 60° C. and stirred for 24 hours, such that epoxy groups of the modified PES and DS reacted to perform a ring-opening reaction to form a resin. The hollow fiber of the resin was then washed by de-ionized water to neutralize the hollow fiber. Subsequently, the hollow fiber of the resin was dipped into deionized water to store the hollow fiber. As known from titration method, the modified PES and DS in the resin have a weight ratio of 100:20. The hollow fiber had a porosity of 89%, which was determined by calculating XCT (X-ray Computed Tomography) images and SEM cross-sectional images.

### Example 8

80 hollow fibers of the resin in Example 7 (having a length of 25 cm) were put into a glass tube to form a bundle. Two terminals of the bundle were sealed with epoxy resin to fix the hollow fibers, and two cross-sections of the fibers were exposed. 130 mL of porcine serum (commercially available from Gibco™) flowed through the bundle at a flow rate of 50 mL/min and circulated to reflow through the bundle for a total 4 hours. The clear liquid was then taken to analyze the composition of the treated porcine serum (Measured by Direct Homogeneous Surfactant (ADVIA 1800, ADVIA Chemistry XPT, Dimension EXL)), as tabulated in Table 2.

The above test was repeated, and the difference was the hollow fibers being changed to the hollow fibers of PES in Comparative Example 1.

The above test was repeated, and the difference was the hollow fibers being changed to the hollow fibers of PES and DS mixture in Comparative Example 2.

The above test was repeated, and the difference was the hollow fibers being changed to the hollow fibers of the modified PES in Example 3.

TABLE 2

| | TCO (mg/dL) | HDL (mg/dL) | LDL-C (mg/dL) | TG (mg/dL) |
|---|---|---|---|---|
| Non-treated | 112 | 32 | 62 | 61 |
| porcine serum | | | | |
| Porcine serum after | 94 | 26 | 48 | 52 |
| being treated by the | | | | |
| hollow fiber of the | | | | |
| resin in Example 7 | | | | |
| Porcine serum after | 112 | 32 | 62 | 61 |
| being treated by the | | | | |
| hollow fiber of PES in | | | | |
| Comparative Example 1 | | | | |
| Porcine serum after | 112 | 32 | 62 | 61 |
| being treated by the | | | | |
| hollow fiber of PES | | | | |
| and DS mixture in | | | | |
| Comparative Example 2 | | | | |
| Porcine serum after | 112 | 32 | 62 | 61 |
| being treated by the | | | | |
| hollow fiber of the | | | | |
| modified PES in | | | | |
| Example 3 | | | | |

As shown in Table 2, only the hollow fiber of the resin (formed by reacting the modified PES and DS) could lower the LDL-C concentration in the porcine serum, while the hollow fibers of PES, the mixture of PES and DS, and the modified PES could not lower LDL-C concentration in the porcine serum.

### Example 9

The cell viability of the film of the resin in Example 4 and the hollow fiber of the resin in Example 7 was tested according to the standard ISO 10993. The cell viability of the film in Example 4 was 104±5%, and the cell viability of the hollow fiber in Example 7 was 98±5%. Obviously, the adsorption material formed by reacting the modified PES and DS was bio-compatible.

It will be apparent to those skilled in the art that various modifications and variations can be made to the disclosed methods and materials. It is intended that the specification and examples be considered as exemplary only, with the true scope of the disclosure being indicated by the following claims and their equivalents.

What is claimed is:

1. A resin, formed through a ring-opening reaction of dextran sulfate sodium salt and a modified polyether sulfone,
wherein the modified polyether sulfone is formed by a reaction of polyether sulfone and an epoxy compound containing a carbon-carbon double bond.

2. The resin as claimed in claim 1, wherein the polyether sulfone and the epoxy compound containing a carbon-carbon double bond have a weight ratio of 100:20 to 100:30.

3. The resin as claimed in claim 1, wherein the modified polyether sulfone and the dextran sulfate sodium salt have a weight ratio of 100:1 to 100:1500.

4. The resin as claimed in claim 1, wherein a repeating unit of the polyether sulfone is 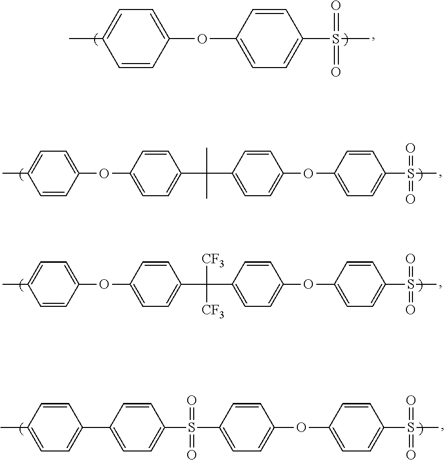
or a combination thereof.

5. The resin as claimed in claim 1, wherein the polyether sulfone has a weight average molecular weight of 55,000 g/mol to 75,000 g/mol.

6. The resin as claimed in claim 1, wherein the dextran sulfate sodium salt has a weight average molecular weight of 5000 g/mol to 10000 g/mol.

7. The resin as claimed in claim 1, wherein the epoxy compound containing a carbon-carbon double bond is 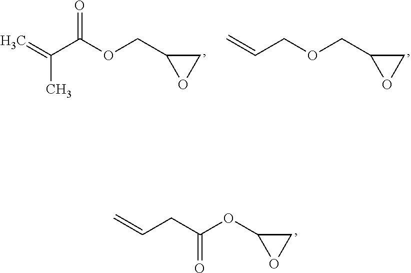
or a combination thereof.

8. An adsorption material, comprising the resin as claimed in claim 1,
wherein the adsorption material is in a form of a porous film or a porous hollow fiber.

9. The adsorption material as claimed in claim 8, wherein the adsorption material has a porosity of 60% to 90%.

10. The adsorption material as claimed in claim 8, wherein the porous film has a thickness of 20 micrometers to 60 micrometers.

11. The adsorption material as claimed in claim 8, wherein the porous hollow fiber has an inner diameter of 300 micrometers to 600 micrometers, and the porous hollow fiber has a thickness of 80 micrometers to 150 micrometers.

12. A method of lowering low-density lipoprotein cholesterol concentration in blood, comprising:
bringing blood into contact with the adsorption material as claimed in claim 8 to lower low-density lipoprotein cholesterol concentration in the blood.
